(19) United States
(12) Patent Application Publication
(10) Pub. No.: US 2026/0217905 A1
(43) Pub. Date: Jul. 30, 2026

(54) PROCESS FOR MANUFACTURING LACTIDE

(71) Applicant: Purac Biochem B.V., Gorinchem (NL)

(72) Inventors: Rens VENEMAN, Gorinchem (NL); Taco Pascal VAN DOORN, Gorinchem (NL)

(21) Appl. No.: 19/578,398

(22) Filed: Mar. 25, 2026

Related U.S. Application Data
(63) Continuation of application No. PCT/EP2024/076884, filed on Sep. 25, 2024.

(30) Foreign Application Priority Data
Sep. 26, 2023 (EP) 23199858.4

(51) Int. Cl. C08G 63/08; B01D 9/00; C07D 319/12

(52) U.S. Cl. CPC C08G 63/08; B01D 9/00; C07D 319/12; B01D 2009/0086

(57) ABSTRACT

The invention pertains to a process for manufacturing lactide with a reduced content of non-lactide contaminants comprising
providing a lactide feed comprising lactide and non-lactide contaminants, the lactide comprising 50-85 wt. % (calculated on total lactide) of meso-lactide and 50-15 wt. % (calculated on total lactide) of L-lactide, and
subjecting said feed to a joint crystallisation step to generate a liquid stream comprising non-lactide contaminants and a solid lactide stream reduced in non-lactide contaminants, which solid lactide stream comprises 50-85 wt. % (calculated on total lactide) of meso-lactide and 50-15 wt. % (calculated on total lactide) of L-lactide.

The process of the present invention provides lactide with a low content of non-lactide contaminants, while lactide is recovered in a high yield. The process can be embedded in lactide manufacturing and purification sequences, and in the manufacture of PLA.

### CROSS REFERENCE TO RELATED APPLICATIONS

The present application is a continuation of International Application No. PCT/EP2024/076884, filed Sep. 25, 2024, which claims priority to European Patent Application No. 23199858.4, filed Sep. 26, 2023, all of which are hereby incorporated by reference herein in their entireties.

The invention pertains to a process for manufacturing lactide from lactic acid. In particular, the invention pertains to a process for manufacturing lactide from lactic acid which in a cost- and apparatus efficient manner generates lactide fractions which are suitable for further processing.

Lactide, the cyclic dimer of lactic acid, is well known in the art as a starting material for polylactide polymer, which is also indicated as polylactic acid or PLA, and for lactide copolymers. PLA and lactide copolymers are used in medical applications, for example in biodegradable sutures, clamps, bone plates, and biologically active controlled release devices. Additionally, PLA is an attractive polymer for many technical applications, e.g., in packaging, because it is biodegradable and can be obtained from renewable resources.

There are three stereochemical forms of lactide, namely L-lactide, which consists of two L-lactic acid moieties, D-lactide, which consists of two D-lactic acid moieties, and meso-lactide, which consists of a L-lactic acid moiety and a D-lactic acid moiety. The equimolar blend of L-lactide and D-lactide is also referred to as racemic (or rac-) lactide or D,L-lactide.

The relative amount of different monomers used in polymerization influences the properties of the resulting polymer, in particular PLA, including its crystallization kinetics and melting point. It is therefore conventional to regulate the relative amounts of the different lactide stereoisomers in PLA. L-lactide and D-lactide are each other's enantiomers, and have the same physical properties such as melting point and boiling point, and thus also the same distillation behaviour. Meso-lactide has different physical properties and is more volatile than either L- or D-lactide.

Conventionally, lactide is manufactured from lactic acid by a process comprising the steps of oligomerising lactic acid to form lactic acid oligomers, and depolymerising the lactic acid oligomers in the presence of a catalyst, to form lactide. The lactic acid can be obtained from many sources, e.g., by subjecting a hydrocarbon source to a fermentation medium to manufacture lactic acid, followed by isolating the lactic acid. Generally, lactic acid obtained from industrial fermentation processes is highly enriched in L-lactic acid. The lactide obtained from the oligomerisation/depolymerisation reaction sequence is subjected to a number of separation steps to remove contaminants such as water and carboxylic acids, and to generate lactide fractions with a high enantiomeric purity, to allow separate processing of the different isomers.

An integrated process for manufacturing PLA starting from lactic acid is described, e.g., in Henton (Natural Fibers, Biopolymers, and Biocomposites, Edited By Amar K. Mohanty, Manjusri Misra, Lawrence T. Drzal, ISBN 9780849317415, Published Apr. 8, 2005 by CRC Press, Chapter 16, Polylactic Acid Technology; D. E. Henton, P. Gruber, J. Lunt, and J. Randall). Lactic acid obtained through fermentation is converted to a prepolymer through condensation polymerisation. The prepolymer is then converted to lactide. The lactide is subjected to a first distillation step, intended in particular to remove water and lactic acid monomer. The lactide is then subjected to a second distillation step, in which meso-lactide is separated, resulting in a meso-lactide stream and an L-lactide stream. Portions of the two streams can be combined and provided to the polymerisation step where PLA is formed through ring-opening polymerisation.

As is well known in the art, the presence of contaminants in the feed of a polymerisation process is often associated with a detrimental effect on polymer properties. For many polymers the presence of contaminants has been shown to lead to undesirable yellowing of the polymer. The presence of contaminants may also affect further properties of the polymer and its production, e.g., its polymerization kinetics, its molecular weight and suitability for applications intended for direct food contact. To address this problem, the meso-lactide stream and the L-lactide stream are generally subjected to further purification steps.

US2016/068505 describes a process in which a lactide crude is subjected to a first distillation step to remove volatile contaminants such as water and lactic acid. The lactide-containing side fraction may contain 90-99.5 wt %, preferably 95-99 wt % of L-lactide and 0-10 wt %, preferably 0-5 wt % of meso-lactide. This lactide stream is subjected to melt crystallisation to recover a purified L-lactide stream and a drain stream containing L-lactide and meso-lactide. The drain stream may be combined with a stream containing meso-lactide derived from the distillation step, and the combined stream is subjected to further distillation. WO2023/036948 describes a process for purifying meso-lactide through solvent crystallisation.

CN114133374 describes melt crystallisation of L-lactide, or D-lactide, or DL-lactide to remove non-lactide contaminants.

U.S. Pat. No. 6,310,218 describes the use of melt-crystallisation to separate meso-lactide from D- or L-lactide, making use of the non-eutectic crystallisation behaviour of lactide mixtures containing large amounts of L-lactide (or D-lactide) and minor amounts of meso-lactide.

Minimizing losses and maximizing lactide yield in these purification steps is crucial for the economic feasibility of the process. There is need in the art for a process for manufacturing lactide which provides lactide with a low content of non-lactide contaminants, while lactide is recovered in a high yield. The present invention provides such a process.

The invention pertains to a process for manufacturing lactide with a reduced content of non-lactide contaminants, which comprises the steps of
providing a lactide feed comprising lactide and non-lactide contaminants, the lactide comprising 50-85 wt. % (calculated on total lactide) of meso-lactide and 50-15 wt. % (calculated on total lactide) of L-lactide,
subjecting said feed to a joint crystallisation step to generate a liquid stream comprising non-lactide contaminants and a solid lactide stream reduced in non-lactide contaminants, which solid lactide stream comprises 50-85 wt. % (calculated on total lactide) of meso-lactide and 50-15 wt. % (calculated on total lactide) of L-lactide.

It is a feature of the present invention that a joint crystallisation step is carried out, in which both meso-lactide and L-lactide are allowed to crystallise from the feed. This joint crystallisation step allows efficient separation of lactide species from non-lactide contaminants. The purified lactide stream containing both meso-lactide and L-lactide can be processed as desired, without necessarily being separated to meso-lactide and L-lactide.

Purification steps known in the field are focussed on either recovering L-lactide and D-lactide from a process stream containing L-lactide, D-lactide, meso-lactide and contaminants, or recovering meso-lactide from a process stream containing L-lactide, D-lactide, meso-lactide and contaminants. In both cases, the non-recovered lactides are discarded as lower-value by-products.

It has unexpectedly been found that it is possible to selectively crystallise L-lactide and meso-lactide in combination from a feed which contains structurally related compounds such as lactic acid and lactic acid oligomers. This makes it possible to obtain a lactide product with a high lactide purity and a low content of lactic acid and lactic acid oligomers from a feed stream comprising L-lactide, meso-lactide and non-lactide contaminants, while such feed streams in conventional lactide processes are often regarded as waste streams. The incorporation of this process thus results in an increase in lactide yield of the overall process at a relatively low cost.

The process of the present invention makes use of the fact that a mixture of L-lactide and meso-lactide with a specific composition shows eutectic crystallisation behaviour. This makes it possible to jointly crystallise both meso-lactide and L-lactide, to separate these species from contaminants, in particular acid-containing contaminants. This is different from the processes in the prior art, where crystallisation steps are used to separate one lactide isomer, generally L-lactide, from a mixture of lactide isomers.

In one embodiment, the present invention pertains to a process wherein the joint crystallisation step according to the invention is used as one step in a process for purifying a lactide feed.

The present invention also pertains to a process for manufacturing lactide from lactic acid in which the process as specified above is one of the steps.

The invention further pertains to a process for manufacturing lactide-containing polymers, for example PLA, wherein the stream comprising 50-85 wt. % of meso-lactide and 50-15 wt. % of L-lactide reduced in non-lactide contaminants is used in the manufacture of lactide-containing polymers, for example PLA.

In another aspect the invention relates to a process for producing lactic acid, wherein at least a portion of the stream comprising 50-85 wt. % of meso-lactide and 50-15 wt. % of L-lactide reduced in non-lactide contaminants is subjected to a hydrolysation step.

In a further aspect, the invention pertains to a process in which the stream comprising 50-85 wt. % of meso-lactide and 50-15 wt. % of L-lactide is derived from a PLA recycle stream. PLA recycle streams, whether derived from a PLA manufacturing process or from PLA obtained from the market. e.g., from PLA-containing packaging materials, can have a widely varying composition, both with respect to the stereochemical forms of the lactide and with respect to the nature of the non-lactide contaminants, and the process according to the invention can find application in maximising the recovery of lactide values from these starting materials. If so desired, intermediate purification steps can be carried out. PLA in this context has a molecular weight of at least 1 kg/mole.

Further advantages of the present invention and various embodiments thereof will become apparent from the further specification.

Where appropriate, the invention will be elucidated with reference to the figures, without being limited thereto or thereby.

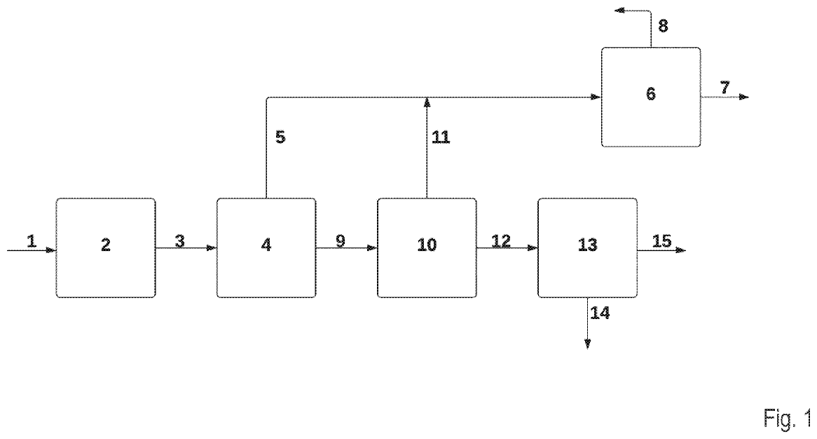

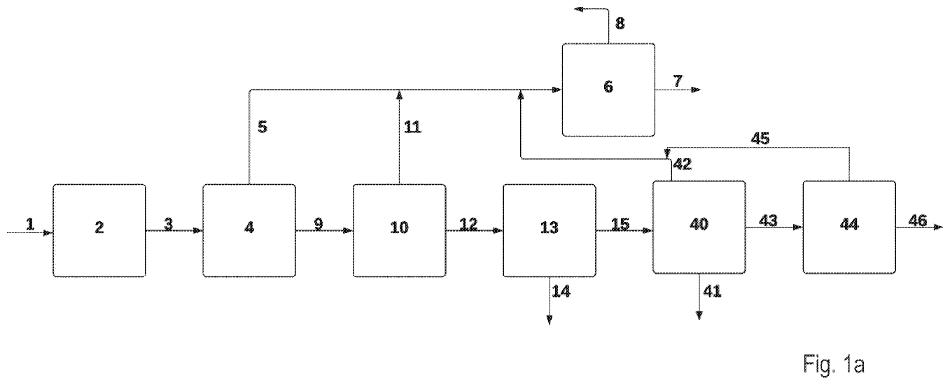

FIG. 1 illustrates a first embodiment of the present invention.

FIGS. 1a and 1b illustrate variations on FIG. 1.

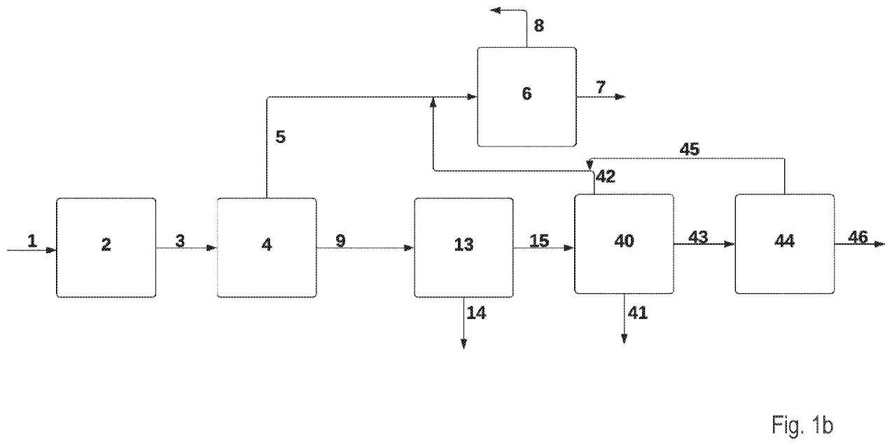

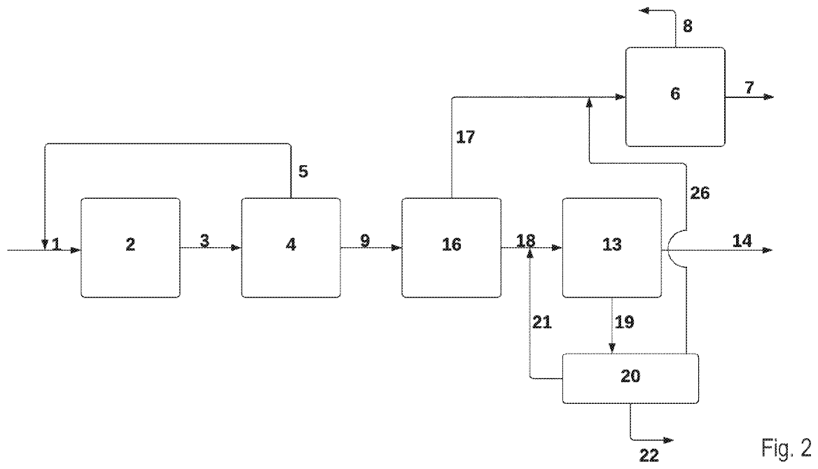

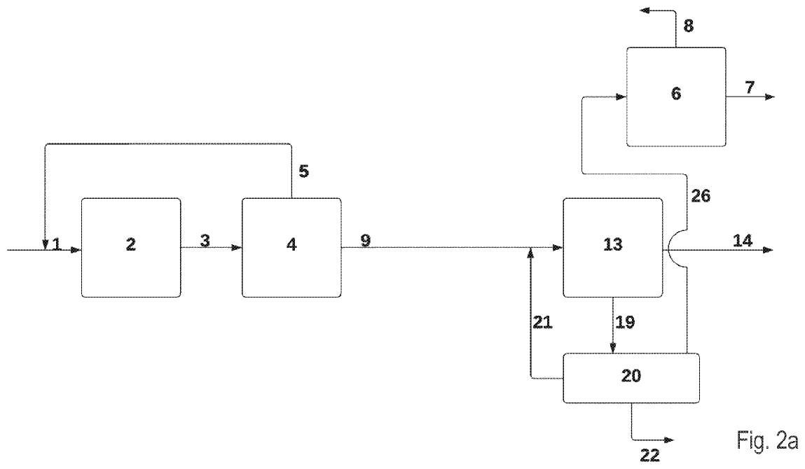

FIG. 2 illustrates a further embodiment of the present invention.

FIG. 2a illustrates a variation on FIG. 2.

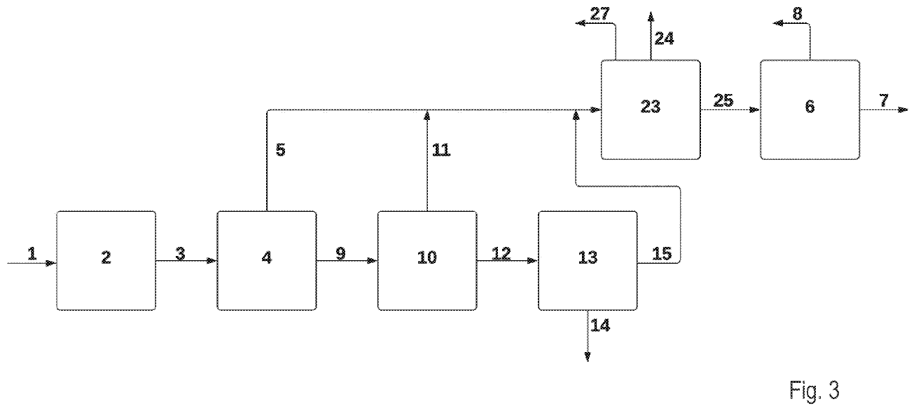

FIG. 3 illustrates a still further embodiment of the present invention.

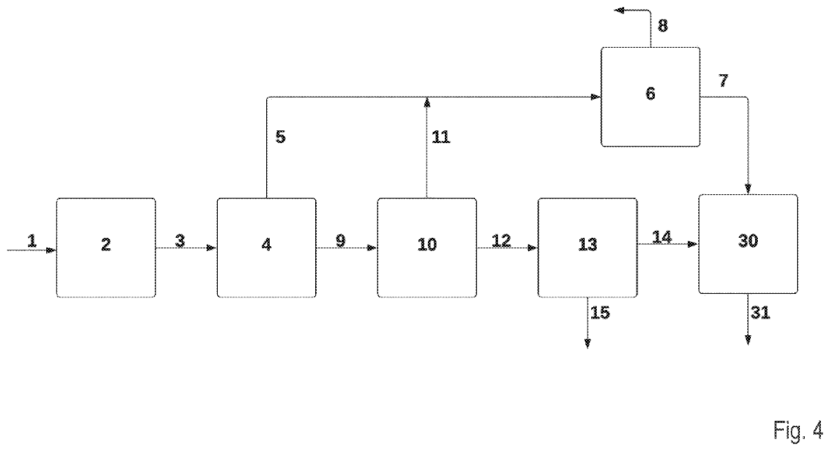

FIG. 4 illustrates a process for manufacturing lactide-containing polymer according to the invention, in which use is made of the joint crystallisation step described herein.

The invention pertains to a process for manufacturing lactide with a reduced content of non-lactide contaminants, which comprises the steps of
providing a lactide feed comprising lactide and non-lactide contaminants, the lactide comprising 50-85 wt. % (calculated on total lactide) of meso-lactide and 50-15 wt. % (calculated on total lactide) of L-lactide,
subjecting said feed to a joint crystallisation step to generate a liquid stream comprising non-lactide contaminants and a solid lactide stream reduced in non-lactide contaminants, which solid lactide stream comprises 50-85 wt. % (calculated on total lactide) of meso-lactide and 50-15 wt. % (calculated on total lactide) of L-lactide.

The feed to be provided to the joint crystallisation step contains lactide and non-lactide contaminants, with the joint crystallisation step being intended to separate at least part of the non-lactide contaminants from the lactide. In general, the feed provided to the crystallisation step will contain 70-99.5 wt. % of total lactide, calculated on the total weight of the stream. The yield of the crystallisation step will decrease with decreasing lactide content in the feed. When the lactide content in the feed is below 70 wt. %, other processes will become more attractive. If the lactide content is above 99.5 wt. %, further purification may not be required. It may be preferred for the feed provided to the crystallisation step to contain at least 80 wt. % of total lactide, in particular at least 85 wt. %, in some embodiments at least 90 wt. %. From a process economy point of view the process may be particularly attractive if the feed provided to the crystallisation step contains at most 99 wt. % of total lactide, in particular at most 98 wt. %.

In one embodiment, the feed contains acidic contaminants, including lactic acid, but possibly also further acidic components. In one embodiment, the feed to be provided to the crystallisation step has a free acid content of at least 30 meq/kg. The free acid content can be determined by means of titration using for instance sodium methylate or potassium methylate in water-free methanol.

The free acid content depends on the source of the feed, and may be much higher than 30 meq/kg. In fact, the process of the present invention may be particularly attractive for feeds with a higher acid content, as the joint crystallisation step of the present invention allows efficient processing of such feeds. Therefore, in some embodiments, the free acid content of the feed that is provided to the joint crystallisation step is at least 50 meq/kg, preferably at least 80 meq/kg, in particular at least 120 meq/kg, or at least 150 meq/kg. The upper limit is not critical. A value of 700 meq/kg may be mentioned as upper limit. In general, the free acid content will be lower, e.g., at most 400 meq/kg, in particular at most 300 meq/kg, often at most 200 meq/kg.

Depending on the source of the feed to be provided to the joint crystallisation step, it may contain various further components. Examples of further components from which the lactide is to be separated include lactic acid and lactic acid oligomers, acidic components such as formic acid, acetic acid, succinic acid, pyruvic acid, and several different hydroxy-acids and alcohols. The process according to the invention is particularly suitable for separating lactide from lactic acid, lactic acid oligomers and other (hydroxy) acids that are typical by-products of lactic acid fermentation.

The lactide in the feed to be provided to the joint crystallisation step comprises 50-85 wt. % of meso-lactide and 50-15 wt. % of L-lactide, calculated on the total amount of lactide species. The lactide may further comprise D-lactide, but the amount thereof will generally be low, e.g., in the range of 0.01 to 5 wt. % (based on the total weight of lactide in the feed). It is noted that D-lactide, if present, will follow the L-lactide. Accordingly, this component will not be discussed separately.

The joint crystallisation step as carried out in the present invention is intended to crystallise both meso-lactide and L-lactide, to separate these species from contaminants, in particular acid-containing contaminants. To allow joint crystallisation of meso-lactide and L-lactide it is necessary that the relative amounts of the two species are such that they can both crystallise from the feed in a joint crystallisation step. The eutectic point of a meso-lactide/L-lactide crystallisation is at a meso-lactide content of 68%. Accordingly, the amount of meso-lactide should be in the range of 50-85 wt. %, with the amount of L-lactide being in the range of 50-15 wt. %. Depending on the source of the feed and the intended purpose, it may be preferred for the feed to have a meso-lactide content in the range of 55-80 wt. %, in particular 60-75 wt. %, with the L-lactide content being in the range of 45-20 wt. %, in particular 40-25 wt. %, in some embodiments 40-35 wt. %.

In the context of the present specification, a joint crystallisation step is a step in which both meso-lactide and L-lactide crystallise in solid form in the same step. As indicated above, the feed to the joint crystallisation step will have a meso-lactide content in the range of 50-85 wt. %, with the amount of L-lactide being in the range of 50-15 wt. %. The joint crystallisation step can follow directly after a crystallisation step in which only a single lactide isomer crystallises. It can even be carried out in the same unit. For example, if a feed is provided which contains 80 wt. % of L-lactide and 20 wt. % of meso-lactide, calculated on total lactide. First L-lactide will crystallise from the system upon cooling of the system, reducing the L-lactide content of the liquid fraction.

Once the composition of the liquid fraction has reached an L-lactide content of below 50 wt. % joint crystallisation will begin. After reaching the desired end temperature of the joint crystallisation step the solid phase formed will contain both L-Lactide and meso-lactide. For reasons of process efficiency it may be preferred to carry out the joint crystallisation step in a separate unit, without being preceded by an L-lactide crystallisation step in the same unit.

The feed to be subjected to joint crystallisation is provided in the liquid state. Depending on the composition of the feed and the preceding process steps, it is generally at a temperature of at least 50° C., in particular at least 54° C., in some embodiments at least 58° C. This will also depend on the composition of the feed. As a maximum, a value of 80° C. may be mentioned.

The joint crystallisation can be carried out by solution crystallisation or by melt crystallisation.

In one embodiment, the joint crystallisation is carried out through solution crystallisation. In solution crystallisation, a solution of lactide in solvent is provided, which is then brought to crystallisation conditions, i.e., conditions at which lactide, in the present case a combination of L-lactide and meso-lactide crystallises from the feed. Crystallisation may be promoted by decrease in temperature, by removal of solvent, or by the combination thereof, e.g., through solvent evaporation.

The solvent to be used in solution crystallisation may be any solvent in which the lactide can dissolve and from which the lactide can crystallise. In one embodiment, a solvent is used which comprises at least 70 wt. % of at least one compound selected from the group of ketones of the formula R1-C(═O)—R2, wherein R1 and R2 are independently selected from methyl, ethyl, propyl, isopropyl, n-butyl, sec-butyl, t-butyl, and isobutyl and ethers of the formula R3-O—R4, wherein R3 and R4 are independently selected from methyl, ethyl, propyl, isopropyl, n-butyl, sec-butyl, t-butyl, and isobutyl. It is preferred for the solvent to consist for at least 80 wt. %, more in particular at least 90 wt. %, still more in particular at least 95 wt. %, even more in particular at least 98 wt. % of the total of at least one compound selected from the group of ketones and ethers as specified above. It has been found that these compounds are particularly attractive as solvents since they combine good solubility properties with low reactivity and good crystallisation and separation properties.

It is preferred for the solvent to comprise less than 5 wt. %, in particular less than 2 wt. %, more in particular less than 1 wt. % of each of alcohols, esters, and aromatic solvents. The presence of alcohols and esters is disadvantageous because they may react with the lactide or contaminants present in the mixture. The presence of aromatic solvents is disadvantageous in view of environmental and food contact concerns associated therewith.

Where ketones are used of the formula R1-C(═O)—R2, wherein R1 and R2 are independently selected from methyl, ethyl, propyl, isopropyl, n-butyl, sec-butyl, t-butyl, and isobutyl, it is preferred for R1 to be methyl. It is further preferred for R1 to be methyl and R2 to be selected from methyl, ethyl, and isobutyl. It is particularly preferred for both R1 and R2 to be methyl, resulting in dimethylketone or 2-propanone, also known as acetone.

Where ethers are used of the formula R3-O—R4, wherein R3 and R4 are independently selected from methyl, ethyl, propyl, isopropyl, n-butyl, sec-butyl, t-butyl, and isobutyl, it is preferred for R1 to be methyl, ethyl, or isopropyl. It is particularly preferred for R3 and R4 to be independently selected from methyl, ethyl, or isopropyl. Diisopropylether may be mentioned as preferred. The use of ketones has been found to be preferred over the use of ethers.

As indicated above, in one embodiment the solvent comprises a total of at least 70 wt. % of at least one compound selected from the group of ketones and ethers as specified herein. While the use of mixed solvents of various compounds within this group is possible in principle, it is generally not considered necessary or preferred. Accordingly, in one embodiment, the solvent comprises a total of at least 70 wt. % of a single compound selected from the group of ketones and ethers as specified herein, preferably at least 80 wt. %, more in particular at least 90 wt. %, still more in particular at least 95 wt. %, even more in particular at least 98 wt. %.

The amount of solvent used in solution crystallisation may vary within wide ranges, e.g., in the range of 2-60 wt. %, also depending on the nature of the solvent, calculated on the solution to be subjected to solution crystallisation. However, if a large amount of solvent is used, deep cooling may be required to obtain crystallisation. Additionally, equipment size will increase when large amounts of solvent are used. Conversely, if very little solvent is used, the amount of lactide which can dissolve in the solvent may be so low that a meaningful process may not be obtained.

If the solubility of the lactide in the solvent so allows, it may be preferred for reasons of process efficiency to use less solvent, e.g., at most 50 wt. %, in some embodiments at most 40 wt. % solvent, in particular at most 35 wt. %, in some embodiments at most 30 wt. %. The lower limit is governed by the solubility of the lactide in the solvent. To obtain adequate dissolution, crystallisation and separation behaviour it may be preferred for the solvent content to be at least 2 wt. %, in particular at least 5 wt. %, in some embodiments at least 10 wt. %.

The exact amount of solvent will depend on the nature of the solvent, viz, the solubility of lactides in the solvent, and the temperature of the mixture of solvent and lactide. A higher temperature is accompanied by an increased solubility of lactide in the solvent, resulting in less solvent being required to obtain a solution of lactide in the solvent.

As will be evident to the skilled person, the presence of substantial amounts of water in the system is to be avoided, as water promotes the hydrolysis of lactide to lactic acid. Accordingly, it is preferred to use water-free solvents, and to otherwise take steps necessary to prevent the presence of water.

In one embodiment, the temperature of the solution is reduced, e.g., to a value below 50° C., in particular below 45° C., more in particular below 40° C., still more in particular below 35° C., even more in particular below 30° C. In one embodiment the temperature is reduced to a value below the eutectic point. From an industrial point of view working below 0° C. may not be practical. A lower limit of 5° C., in particular 10° C. may be mentioned. As will be evident to the skilled person, the temperature will influence crystal yield with a higher yield being obtained at lower temperatures. The suitable temperature will depend on the nature and amount of the solvent and on the desired crystal yield for the individual crystallisation step.

If so desired, the lactide crystals recovered from the solvent may be washed with solvent, to remove impurities adhering to the crystals. To prevent lactide dissolving in the washing solvent, the washing solvent is generally relatively cold.

In one embodiment, the crystallisation step is a solution crystallisation step carried out as suspension crystallisation, followed by recovery of the crystals by filtration, preferably on a belt filter, followed by washing of the crystals, preferably also on a belt filter. Another preferred method for the solid liquid separation step is centrifugal filtration, where mother liquor may be efficiently separated from the crystals.

Where solution crystallisation is carried out, it has been found that the solvent content of the crystals obtained in the process according to the invention may be relatively low, e.g., below 1 wt. %, in particular below 0.7 wt. %, more in particular below 0.5 wt. %. As a result of the relatively high volatility of the solvents, the solvent content in the crystals can easily be reduced further by evaporating the solvent, e.g., under sub-atmospheric pressure.

If a solution crystallisation is applied, it is possible to purify the solvent, e.g., through distillation. Therefore, in one embodiment, the process encompasses the step of subjecting used solvent to a purification step, e.g., a distillation step, and providing purified solvent to a lactide crystallisation step.

The crystallisation of the lactide may also be carried out through melt-crystallisation, i.e., crystallisation in the absence of solvent, i.e., at a solvent content of less than 2 wt. %, in particular less than 1 wt. %. In melt crystallisation, the crystallisation is effected by reducing the temperature of a lactide melt to a value which is such that lactide crystallises from the lactide melt. This can be done, e.g., using methods known in the art, such as by passing the lactide melt over a cooled surface. The temperature at which melt crystallization occurs is dependent on the melting temperature of the compound whereas in solvent crystallization the temperature at which crystallization occurs depends on the solubility of the compound in the particular solvent. To effect melt crystallisation, the temperature of the melt is generally reduced to a value of e.g., below 50° C., in particular below 45° C., more in particular below 40° C., still more in particular below 35° C., even more in particular below 30° C. In one embodiment the temperature is reduced to a value below the eutectic temperature of the mixture of L-lactide and meso-lactide. From an industrial point of view working below 0° C. may not be practical. A lower limit of 5° C., in particular 10° C. may be mentioned.

Crystallisation may be promoted by the addition of seed crystals.

The joint crystallisation step in the process according to the invention may be a single crystallisation step. It is also possible to carry it out in a number of individual sequential stages. If so desired, the crystallisation step can be carried out in 2-10 sequential crystallisation stages.

This could include re-crystallization of the solids of a particular step and exhaustion steps in which the mother liquor of a previous step is crystallized again to improve the overall yield.

In one embodiment, the crystallisation stages are carried out in such a way that at least 15% of the lactide provided to a stage crystallises in solid form in that stage. The optimum amount will depend on the process and the ability to still efficiently separate the mother liquor containing the impurities from the solid lactide. In general, crystallising lower amounts may lead to larger crystals with preferred filtration and washing properties and accordingly higher purities. On the other hand, higher amounts may be associated with higher overall process efficiency. In one embodiment, at least one, preferably all of the crystallisation stages are carried out such that at least 20% of the lactide in the feed crystallises in solid form, in particular at least 25%, more in particular at least 30%. Conversely, at high crystallization levels of above 80% slurries may become too viscous to be effectively stirred and pumped, crystal attrition may occur and liquid solid separation may become less effective. Accordingly, it may be preferred to crystallise at most 80% of the lactide in a single crystallisation stage. The preferred level of crystallisation in a specific embodiment will also depend on the process configuration, and on whether or not a solvent is present. If more than one crystallisation stage is carried out, the amount of lactide crystallised in an individual stage may be reduced.

Where a number of sequential solution crystallisation stages are carried out, solvent recovered from a later crystallisation stage may be recycled to an earlier crystallisation stage. This is possible because the solvent recovered from a later crystallisation stage has a lower contaminant content than solvent recovered from earlier crystallisation stages.

For a high yield overall process, further lactide may be recovered from solvents discarded from the crystallisation steps. This may come at the cost of overall purity and the final process will be chosen such that a good balance between yield and quality is obtained.

Depending on the type of crystallisation, suitable apparatus include the use of a static crystalliser, a scraped-wall crystallizer and a stirred vessel. Stirred vessel suspension crystallizations may be cooled by external wall cooling, or evaporative cooling, or both. Suitable apparatus is known to the skilled person and requires no further specification here. Solution crystallisation in a stirred vessel is considered preferred, also in view of the good filtration properties of the lactide crystals formed. The crystals can be recovered though centrifugation, but recovery through filtration is considered preferred. The use of a belt filter may be particularly attractive.

Depending of the amount of lactide that crystallises in the crystallisation step or stage, the mother liquor separated from the solid lactide can still contain substantial amounts of lactide. The mother liquor can be processed as desired. The mother liquor can be reheated and provided to a further crystallisation step or stage, which may be a solution crystallisation step or a melt crystallisation step. The mother liquor can also be purged from the process, or hydrolysed to provide a technical grade lactic acid. How the mother liquor is handled will depend on the amount of lactide present therein, but also on the amount of contaminants. Obviously, if the amount of contaminants in the mother liquor is too high, recycle thereof to the crystallisation process may lead to the addition rather than the reduction of contaminants in the system, which should be avoided.

Depending on the contaminant content of the lactide, it may be desirable to subject the solid product of the crystallisation step to a re-crystallisation step, to further reduce the contaminant content.

The crystallisation can be carried out as a batch process or as a continuous process.

The crystallisation step(s) yield a solid lactide product. In general, the lactide resulting from the crystallisation has a lactide content of at least 95 wt. %, in particular at least 98 wt. %, more in particular at least 99 wt. %.

The aim of the crystallisation step in the process according to the invention is to reduce the contaminant content of the lactide, including but not limited to its free acid content. The reduction of the free acid content can be expressed through the free acid reduction factor, which is defined as the free acid content of the starting material divided by the free acid content of the product crystals. A reduction factor of 1 thus means that the free acid content is not reduced. A reduction factor of 2 means that the free acid content has been halved. The crystallisation step in the process according to the invention can often be carried out to achieve a reduction factor of at least 2, in many embodiments at least 3, or at least 4, and in some embodiments even higher, in a single crystallisation step.

Based on the teachings in the present specification it is within the scope of the skilled person to select crystallisation conditions in such a manner that the desired free acid reduction factor can be achieved.

The product from the crystallisation step has a reduced free acid content as compared to the lactide provided to the crystallisation step. Depending on the free acid content of the starting lactide, the product from the crystallisation step generally has a free acid content of at most 20 meq/kg. It is preferred for the free acid content to be at most 15 meq/kg, in particular at most 10 meq/kg. Depending on the specific use of the lactide product, it may be desired for the free acid content to be much lower, e.g., at most 5 meq/kg, at most 2 meq/kg, or at most 1 meq/kg.

As indicated above, if the purity of the crystals obtained after a first crystallisation step is insufficient, the product crystals may be subjected to a recrystallisation step.

The product resulting from the joint crystallisation step comprises both meso-lactide and L-lactide. In general, depending on the conditions at which crystallisation is carried out, the amount of meso-lactide in the product resulting from the joint crystallisation step is in the range of 50-85 wt. %, with the amount of L-lactide being in the range of 50-15 wt. %. Depending on the crystallisation conditions, it may be preferred for the product resulting from the joint crystallisation step to have a meso-lactide content in the range of 55-80 wt. %, in particular 60-75 wt. %, with the L-lactide content being in the range of 45-20 wt. %, in particular 40-25 wt. %, in some embodiments 40-35 wt. %.

The process according to the invention can be embedded into various multistep processes.

In one embodiment of the present invention, the joint crystallisation step discussed above is present in a process for manufacturing lactide from lactic acid. Lactic acid may be obtained from various sources, including fermentation of hydrocarbons and various industrial processes including degradation and recycling of polylactic acid.

The present invention thus also pertains to a process for manufacturing lactide from lactic acid, which comprises the steps of
reacting lactic acid to form lactide,
providing a lactide feed comprising lactide and non-lactide contaminants, the lactide comprising 50-85 wt. % (calculated on total lactide) of meso-lactide and 50-15 wt. % (calculated on total lactide) of L-lactide,
subjecting said feed to a joint crystallisation step to generate a stream containing non-lactide contaminants and a solid lactide stream reduced in non-lactide contaminants, which lactide stream comprises 50-85 wt. % (calculated on total lactide) of meso-lactide and 50-15 wt. % (calculated on total lactide) of L-lactide.

It is possible to obtain lactide directly from lactic acid. It is more conventional, however, for the reaction of lactic acid to lactide to be carried out in oligomerisation/depolymerisation process, comprising the steps of
subjecting lactic acid to condensation oligomerisation to form low molecular weight poly(lactic acid), and
depolymerising the low molecular weight poly(lactic acid) to form lactide.

The steps of obtaining lactide directly from lactic acid, and the steps of reacting lactic acid to form low molecular weight poly(lactic acid) by condensation oligomerisation, and depolymerising the low molecular weight poly(lactic acid) to form lactide are in themselves known in the art. They require no elucidation here. The following may be noted:

In the condensation oligomerisation step a low-molecular weight poly(lactic acid) is formed by condensation oligomerisation of lactic acid. The lactic acid is often obtained from a biological process and generally has a high optical purity. Depending on the source it may contain at least 90% of L-lactic acid, in particular at least 95% L-lactic acid, more in particular at least 98 wt. %. The condensation oligomerisation may be carried out as is known in the art. It generally involves subjecting lactic acid to sub-atmospheric pressure, e.g. 50-500 mbar at elevated temperatures, e.g., 100-200° C., to induce polymerisation by removal of water. The average degree of polymerisation obtained is generally between 5 and 20.

In the depolymerisation step the low-molecular weight polylactic acid is subjected to a depolymerisation step, to convert the low molecular weight polylactic acid to lactide.

Depolymerisation is also known in the art. It is generally carried out in the presence of a catalyst. Metal-containing catalysts are often used, in particular catalysts based on tin, zinc, aluminium, lead, antimony, lead, calcium, and magnesium, e.g., in the form of halide salts or salts of organic acids, such as fatty acids. Tin (II) bis(2-ethyl hexanoate) is often used commercially. Typical concentration of lactide synthesis catalysts may be 20-2000 ppm. Reaction conditions include a temperature of 160 to 260° C., a pressure of 5 to 100 mbar, and a residence time of 10 minutes to 8 hours.

The process according to the invention may be of particular interest when applied to lactide obtained from reworking or recycling of polylactic acid. In the field, the repurposing of polylactic acid is gaining more and more attention. This applies to PLA which has been used, e.g., in the form of packaging material, and has been recovered from the market, and to PLA which is, for some reason not suitable for its intended use. Such PLA generally has an absolute number average molecular weight of above 5 kg/mol, in particular above 10 kg/mol. A maximum value is not critical. A value of 100 kg/mol may be mentioned. Such PLA can be converted to lactide moieties through catalytic depolymerisation. Depending on the composition of the starting PLA and the processing conditions, the stereochemical composition of the lactide product may vary. Additionally, depending on the depolymerisation conditions applied, it may contain relatively large amounts of meso-lactide, lactic acid and/or lactic acid oligomers. Accordingly, the process according to the invention may be of particular value in the processing of lactide feeds derived from reworking or recycling of PLA.

The product from the lactide manufacturing step, either a direct lactide manufacturing step from lactic acid or a depolymerisation step is a lactide stream comprising non-lactide contaminants and meso-lactide, and L and/or D-lactide with L- or D-lactide being the predominant lactide and the other being the non-predominant lactide. Which lactide is the predominant lactide will depend on the stereochemistry of the starting lactic acid. In general, the L-lactide will be the predominant lactide. The crude lactide will also contain water and lactic acid.

The joint crystallisation step can be embedded into a process for manufacturing purified lactide streams. These lactide streams can be streams which are formed in a process for manufacturing lactide from lactic acid. As indicated above, they may also be derived from other sources, e.g., from lactide obtained through hydrolysis of used PLA, or from lactide streams recovered from PLA manufacturing processes.

In one embodiment, the invention pertains to a process for manufacturing a purified lactide stream, which comprises the steps of
providing a lactide composition comprising L-lactide in an amount of 75-99 wt. % (calculated on total lactide), and meso-lactide in an amount of 1-25 wt. % (calculated on total lactide), and non-lactide contaminants, and
generating from that composition a lactide feed comprising lactide and non-lactide contaminants, the lactide in the lactide feed comprising 50-85 wt. % (calculated on total lactide) of meso-lactide and 50-15 wt. % (calculated on total lactide) of L-lactide, and subjecting said feed to a joint crystallisation step to generate a stream containing non-lactide contaminants and a solid lactide stream reduced in non-lactide contaminants, which lactide stream comprises 50-85 wt. % (calculated on total lactide) of meso-lactide and 50-15 wt. % (calculated on total lactide) of L-lactide.

The step of generating the specified lactide feed can be carried out in a number of ways. For example, a separation step can be carried out. Alternatively, a crystallisation step is initiated in which first L-lactide crystallises from the lactide composition, followed by the joint crystallisation.

This process can be incorporated into a process for manufacturing lactide from lactic acid. It can also be incorporated in a process for recovering lactide from other sources.

Where the process is incorporated into the manufacture of lactide from lactic acid, the first step will encompass
reacting lactic acid to form a lactide composition comprising L-lactide in an amount of 75-99 wt. % (calculated on total lactide), and meso-lactide in an amount of 1-25 wt. % (calculated on total lactide), and non-lactide contaminants.

In one embodiment, the invention pertains to a process for manufacturing purified lactide streams which process encompasses the joint crystallisation step described herein, in combination with at least one further crystallisation step, the further crystallisation step being selected from
a crystallisation step in which L-lactide is crystallised from a feed (and meso-lactide is not),
a crystallisation step in which meso-lactide is crystallised from a feed (and L-lactide is not), and
a further joint crystallisation step.

In the present invention, a crystallisation step in which L-lactide is crystallised from a feed (and meso-lactide is not) will be carried out on a lactide composition comprising L-lactide in an amount of 75-100 wt. % (calculated on total lactide), and non-lactide contaminants. Depending on the source, the feed may or may not contain meso-lactide.

A crystallisation step in which meso-lactide is crystallised from a feed (and L-lactide is not) will be carried out on a lactide composition comprising meso-lactide in an amount of 85-100 wt. % (calculated on total lactide), and non-lactide contaminants. Depending on the source, the feed may or may not contain L-lactide.

The concentration ranges provided here are selected such that the cited lactide will crystallise, and other lactide species will not. Nevertheless, depending of the efficiency of the separation between mother liquor (the liquid phase after crystallization) and the solid crystal phase some mother liquor may still be present on/in the solid phase. As a consequence, it may be that small amounts (less than 5 wt. %, preferably less than 2 wt. %, more preferably less than 1 wt. %) of the not cited lactide can still be measured in the solid product of the crystallisation step.

In one embodiment, the invention pertains to a process for manufacturing purified lactide streams which comprises the joint crystallisation step defined herein, in combination with a step in which L-lactide is crystallised from a feed (and meso-lactide is not). This embodiment may be attractive to efficiently recover lactide values from a feed containing both L-lactide and meso-lactide in a cost-effective manner requiring limited use of apparatus.

In one embodiment, the invention pertains to a process comprising the steps of
providing a lactide composition comprising L-lactide in an amount of 75-99 wt. % (calculated on total lactide), and meso-lactide in an amount of 1-25 wt. % (calculated on total lactide), and non-lactide contaminants,
subjecting the lactide composition to a separation step to form a first stream which has an L-lactide content of at least 80 wt. %, calculated on the total weight of the stream, and a second stream comprising lactide and non-lactide contaminants, the lactide in the second stream comprising 50-85 wt. % of meso-lactide and 50-15 wt. % of L-lactide,
subjecting the second stream to the joint crystallisation step described herein, resulting in a stream containing non-lactide contaminants and a lactide stream reduced in non-lactide contaminants, which lactide stream comprises 50-85 wt. % of meso-lactide and 50-15 wt. % of L-lactide (calculated on total lactide).

The first stream which has an L-lactide content of at least 80 wt. % may be treated as desired. In one embodiment, it is subjected to a crystallisation step to generate solid L-lactide.

In this embodiment, the presence of the joint crystallisation step ensures that lactide values are recovered even when they are present in a stream with a low stereochemical purity. This improves the yield of the overall lactide production process. This process can be incorporated into a process for manufacturing lactide from lactic acid. It can also be incorporated in a process for recovering lactide from other sources.

Where the process is incorporated into the manufacture of lactide from lactic acid, the first step will encompass
reacting lactic acid to form a lactide composition comprising L-lactide in an amount of 75-99 wt. % (calculated on total lactide), and meso-lactide in an amount of 1-25 wt. % (calculated on total lactide), and non-lactide contaminants.

There are various ways in which this embodiment can be carried out. In one embodiment, the first stream and the second stream are directly derived from the depolymerisation step. Alternatively, in another embodiment, the product from the depolymerisation step is submitted to a distillation step, and the feed for the joint crystallisation step derives from the distillation step. It is also possible to derive feed for the joint crystallisation step both from the depolymerisation step and from a distillation step. In one embodiment, the L-lactide-containing first stream is separated from the lactide-containing composition through a crystallisation step in which L-lactide is selectively crystallised from the composition.

In the process discussed above, a lactide stream is generated comprising at least 80 wt. % of L-lactide, calculated on the total amount of lactide in the stream. The L-lactide stream may, e.g., have an L-lactide content of at least 85 wt. %, calculated on total lactide, in particular at least 90 wt. %, more in particular at least 95 wt. %, in some embodiments at least 98 wt. %. The L-lactide stream generally has a total lactide content, calculated on the total weight of the stream, of at least 80 wt. %, in particular at least 90 wt. %, in more particular at least 95 wt. %, in some embodiments at least 98 wt. %.

In another embodiment, the invention pertains to a process for manufacturing purified lactide streams which process encompasses
a crystallisation step in which L-lactide is crystallised from a feed (and meso-lactide is not),
a crystallisation step in which meso-lactide is crystallised from a feed (and L-lactide is not), and
a joint crystallisation step.

This embodiment makes it possible to prepare L-lactide and meso-lactide streams of high stereochemical purity, while at the same time using the joint crystallisation step to maximise the recovery of lactide values from the feed.

In one embodiment the invention pertains to a process for providing purified lactide streams, comprising the steps of
providing a lactide composition comprising L-lactide in an amount of 75-99 wt. % (calculated on total lactide), and meso-lactide in an amount of 1-25 wt. % (calculated on total lactide), and non-lactide contaminants,
subjecting said lactide stream to one or more separation steps to generate
a) a stream which has an L-lactide content of at least 80 wt. %, calculated on the total weight of the stream, and subjecting said stream to a crystallisation step to generate solid L-lactide,
b) a stream which has a meso-lactide content of 85-100 wt. % and subjecting said stream to a crystallisation step to generate solid meso-lactide, and
c) a stream comprising 50-85 wt. % (calculated on total lactide) of meso-lactide and 50-15 wt. % of L-lactide 50-85 wt. % (calculated on total lactide), and subjecting said stream to a joint crystallisation step to generate a solid lactide stream comprising 50-85 wt. % (calculated on total lactide) of meso-lactide and 50-15 wt. % of L-lactide 50-85 wt. % (calculated on total lactide).

As above, this process can be incorporated into a process for manufacturing lactide from lactic acid. It can also be incorporated in a process for recovering lactide from other sources.

Where the process is incorporated into the manufacture of lactide from lactic acid, the first step will encompass
reacting lactic acid to form a lactide composition comprising L-lactide in an amount of 75-99 wt. % (calculated on total lactide), and meso-lactide in an amount of 1-25 wt. % (calculated on total lactide), and non-lactide contaminants.

The process according to the invention, including the various embodiments thereof, is particularly attractive for processing lactide on an industrial scale. This is where it is particularly relevant that lactide with high purity can be obtained in an efficient and effective manner from a feed containing impurities such as lactic acid and lactic acid oligomers. Accordingly, in one embodiment of the present invention the joint crystallisation step is carried out in a crystalliser with a volume of at least 0.5 m³, in particular at least 1 m³, more in particular at least 2 m³, in some embodiments at least 5 m³. While not generally limiting, a volume of 20 m³ may be mentioned as a maximum.

The present invention is explained in more detail with reference to the accompanying figures. The figures are for illustration purposes only; the invention is not limited thereto or thereby. The following is noted with respect to the figures:

Embodiments of various figures can be combined unless they are mutually exclusive. The figures are flow sheets illustrating the process according to the invention. The figures do not present a reactor set up. For example, where a separation step is shown in a single step, it may be carried out on more than one reactor. This applies, e.g., to distillation steps and crystallisation steps. Conversely, different steps may be carried out in the same unit. By the same token, the various lines are intended to show how components flow from one reaction step to the other. They do not represent real-life structures. The figures are not intended to show specific engineering features or details, including the design of the various components shown.

The figures do not always show all elements of the process according to the invention. The figures do not show all purge streams or make-up streams that may be present in the practical performance of the process according to the invention although, as will be evident to the skilled person, purge streams and make-up streams may be necessary in practice to maintain stable operation. In addition, auxiliary equipment such as various valves, (vacuum) pumps, heating and cooling equipment, including inlets and outlets for cooling media, analytical devices, control devices and the like are not always shown in the figures, but of course such equipment can be used as necessary or desirable, and is well known to the skilled person.

FIG. 1 illustrates some of the possibilities described above. In FIG. 1, lactic acid is provided through line (1) to oligomerisation unit (2), where it is reacted to form low molecular weight PLA (degree of oligomerisation generally in the range of 5-20). The low-molecular weight PLA is withdrawn through line (3), and provided to depolymerisation unit (4), where lactide is formed. A stream (5) comprising 50-85 wt. % of meso-lactide and 50-15 wt. % of L-lactide, and non-lactide contaminants such as lactic acid is withdrawn from depolymerisation unit (4) and provided to crystallisation unit (6). Depolymerisation unit (4) also generates a stream (9) which is provided to a distillation step (10). Stream (9) comprises 70-95 wt. % L-lactide, in particular 80-90 wt. % L-lactide, and 5-30 wt. % meso-lactide, in particular 10-20 wt. % meso-lactide. The stream may further comprise non-lactide contaminants, generally in an amount of less than 5 wt. %.

In crystallisation unit (6), a mixture of 50-85 wt. % of meso-lactide and 50-15 wt. % of L-lactide is crystallised, and the crystallised product is withdrawn through line (7). Line (8) contains lactic acid and other contaminants.

In FIG. 1, stream (9) is provided to distillation step (10). In distillation step (10) the feed is distilled to generate a first fraction comprising non-lactide contaminants, meso-lactide, and L-lactide, and a second fraction comprising at least 80 wt. % of L-lactide calculated on the total lactide species in the fraction, in particular at least 85 wt. %, more in particular at least 90 wt. %. In the illustrated embodiment, the first fraction is withdrawn through line (11) and provided to crystallisation unit (6). The second fraction, comprising at least 80 wt. % of L-lactide calculated on the total lactide species in the fraction is provided through line (12) to a crystallisation unit (13). In crystallisation unit (13), L-lactide is crystallised from the system in high purity, and removed through line (14). The L-lactide withdrawn through line (14) comprises at least 90 wt. % of L-lactide calculated on the total lactide species, e.g., at least 95 wt. %, in particular at least 98 wt. %, in some embodiments at least 99 wt. %. The liquid effluent from crystallisation unit (13) is withdrawn through line (15).

Thus, FIG. 1 shows how the overall recovery of lactide values from a lactide manufacturing process values can be increased by providing relatively impure side streams generated in the lactide manufacturing process which contain L-lactide, meso-lactide, and non-lactide contaminants to crystallisation unit (6) where it is converted to a product with a high lactide purity.

As indicated above, in one embodiment the process according to the invention encompasses both the combined crystallisation of meso-lactide and L-lactide, and an L-lactide crystallisation step. In one embodiment, these steps are the final processing steps performed on the respective fractions. This is advantageous, because the crystallisation steps yield high purity products containing low amounts of side products, in particular lactic acid and oligomers. This is in contrast with other separation steps such as distillation steps, where the severity of the processing may result in hydrolysis of the product, resulting in the further manufacture of lactic acid monomer and lower lactic acid oligomers. The process preferably also encompasses a crystallisation step for meso-lactide, preferably also as final processing step performed on this fraction.

FIG. 1a shows a variation on the process of FIG. 1, which encompasses three different crystallisations, namely crystallisation of L-lactide, crystallisation of meso-lactide, and crystallisation of L-lactide and meso-lactide in combination. It shows how a number of further low-value side streams can be valorised by providing them to crystallisation unit (6). In FIG. 1a the liquid effluent from crystallisation unit (13) withdrawn through line (15) is provided to a further separation section (40). Separation section (40) may consist, e.g., of one or more interconnected distillation units. Separation section (40) may generate a number of steams. In the illustrated embodiment, it generates an L-lactide stream (41) with a relatively high optical purity. The L-lactide withdrawn through line (41) may comprise, e.g., at least 90 wt. % of L-lactide calculated on the total lactide species, in particular at least 95 wt. %, in particular at least 98 wt. %, in some embodiments at least 99 wt. %. Separation section (40) may also generate a stream (43) rich in meso-lactide, e.g., containing at least 90 wt. % meso-lactide, calculated on the total amount of lactide in the stream. In the figure this stream (43) is provided to crystallisation unit (44), where meso-lactide is crystallised and withdrawn through line (46). Both separation unit (40) and crystallisation unit (44) generate side streams containing L-lactide, meso-lactide, and non-lactide contaminants (streams (42) and (45) respectively). Either or both of these streams may be provided to crystallisation unit (6) for the recovery of lactide values therefrom, as is illustrated in FIG. 1a.

FIG. 1b is a variation on FIG. 1a. In FIG. 1b, stream (9) derived from depolymerisation unit (4) is provided directly to L-lactide crystallisation step (13), without intermediate distillation. This embodiment is attractive when the product obtained from the depolymerisation step has a relatively high L-lactide content and a relatively low contaminant content. It is preferred for the feed provided to crystallisation step (13) to contain. at least 80 wt. % of L-lactide calculated on the total lactide species in the fraction, in particular at least 85 wt. %, more in particular at least 90 wt. %.

FIG. 2 shows a variation on the process presented in FIG. 1. In FIG. 2, stream (5) comprising L-lactide, meso-lactide, and non-lactide contaminants such as lactic acid withdrawn from depolymerisation unit (4) is recycled back to feed stream (1). It is also possible to provide this stream, in part or in its entirety, to the crystallisation unit (6), as in FIG. 1. In FIG. 2, instead of distillation unit (10), a pre-separation unit (16) is provided. Pre-separation unit 16 may be a crystallisation unit or a distillation unit. Pre-separation unit (16) generates a stream (17) containing non-lactide contaminants, meso-lactide, and L-lactide, which is provided to crystallisation unit (6). A stream comprising at least 80 wt. % of L-lactide calculated on the total lactide species in the fraction is withdrawn from pre-separation unit (16) and provided through line (18) to a crystallisation unit (13). L-lactide is crystallised from the system in high purity, and removed through line (14). The mother liquor is withdrawn through line (19), and provided to a further crystalliser (20). This crystalliser generates further lactide which is recycled back to the feed of crystallisation unit (13) though line (21). It also generates a stream (26) containing L-lactide, meso-lactide, and non-lactide contaminants, which is provided to crystalliser (6). Stream (22) is a purge stream.

FIG. 2a is a variation on FIG. 2. In the embodiment provided in FIG. 2a pre-separation unit (16) is dispensed with, and stream (9) derived from depolymerisation unit (4) is provided directly to L-lactide crystallisation step (13), without intermediate distillation. This embodiment is attractive when the product obtained from the depolymerisation step has a relatively high L-lactide content and a relatively low contaminant content. It is preferred for the feed provided to crystallisation step (13) to contain. at least 80 wt. % of L-lactide calculated on the total lactide species in the fraction, in particular at least 85 wt. %, more in particular at least 90 wt. %. As for FIG. 2, stream (5) comprising L-lactide, meso-lactide, and non-lactide contaminants such as lactic acid withdrawn from depolymerisation unit (4) may also be provided, in part or in its entirety, to the crystallisation unit (6), as in FIG. 1.

An attractive feature of this configuration is that it contains no distillation steps.

As compared to the embodiments of FIGS. 1a and 1b, the embodiments of FIGS. 2 and 2a do not contain a separate meso-lactide crystallisation step. Accordingly, the embodiment of FIGS. 2 and 2a may be preferred if the amount of meso-lactide crystallisation step is not desired, e.g., because the amount of meso-lactide present in the feed is relatively limited.

FIG. 3 shows a further variation on the process presented in FIG. 1. In FIG. 3, stream (5) derived from depolymerisation step (4) and stream (11) derived from the distillation step (10) are not provided directly to crystallisation unit (6). Rather, they are provided to a distillation section (23). Stream (15) derived from the L-lactide crystallisation step is also provided to distillation section (23). Distillation section (23) generates an L-lactide stream, which is withdrawn through line (24) and a purge stream withdrawn through line (27). A stream (25) comprising non-lactide contaminants, 50-85 wt. % of meso-lactide (calculated on lactide species in the stream) and 50-15 wt. % of L-lactide (calculated on lactide species in the stream) is provided to crystallisation unit (6), where a mixture of 50-85 wt. % of meso-lactide and 50-15 wt. % of L-lactide is crystallised, and withdrawn through line (7). Distillation section (23) is presented here as a single unit, but it may also encompass a number of sequential distillation units.

The process presented in this figure thus generates three lactide streams, namely the high-purity L-lactide stream (14) derived from L-lactide crystallisation unit (13), the high-purity L-lactide stream (24) derived from distillation section (23), and the high purity mixture of 50-85 wt. % of meso-lactide and 50-15 wt. % of L-lactide withdrawn through line (7). It is an attractive feature of this embodiment that separate separation and crystallisation of meso-lactide is not required.

A particular feature of the claimed invention is that the high purity mixture of 50-85 wt. % of meso-lactide and 50-15 wt. % of L-lactide generated in the joint crystallisation step is suitable for blending with an L-lactide stream to generate a product stream which can be subjected to polymerisation to form polylactide with a desired meso-lactide content.

In one embodiment, therefore, the process according to the invention encompasses the step of combining at least part of the purified mixture of meso-lactide and L-lactide resulting from the joint crystallisation step with a stream comprising at least 75 wt. % of L-lactide, to form a lactide stream comprising 70-99 wt. % L-lactide and 1-30 wt. % meso-lactide, both calculated on total lactide species. Within this embodiment it may be preferred for the lactide fraction in the feed to comprise at least 2 wt. % meso-lactide, in particular at least 3 wt. % meso-lactide, and/or at most 15 wt. % meso-lactide, in particular at most 10 wt. % meso-lactide.

The L-lactide stream preferably comprises at least 85 wt. % of L-lactide, in particular at least 90 wt. % of L-lactide, more in particular at least 95 wt. % of L-lactide, or at least 98 wt. % L-lactide, or at least 99 wt. % L-lactide, calculated as total lactide species. It is preferred for the L-lactide stream to be derived, indirectly or directly, but preferably directly, from an L-lactide crystallisation step.

The combined stream preferably comprises less than 5 wt. % of non-lactide compounds, calculated on the total weight of the stream, in particular less than 4 wt. %, or less than 3 wt. %, or less than 1 wt. %. Preferably the feed has a free acid content of at most 20 meq/kg. It is preferred for the free acid content to be at most 15 meq/kg, in particular at most 10 meq/kg., or at most 5 meq/kg, at most 2 meq/kg, or at most 1 meq/kg.

The preferences given here for the combined stream also apply to the meso-lactide stream and the L-lactide stream.

It is preferred for the combined stream to be provided to a polymerisation process. Accordingly, the present invention also pertains to a process for manufacturing a lactide-based polymer comprising the steps of
providing a lactide stream comprising 50-85 wt. % of meso-lactide and 50-15 wt. % of L-lactide to a polymerisation step in a polymerisation feed, in particular a feed resulting from the joint crystallisation step described herein,
and subjecting the polymerisation feed to a polymerisation step to form a lactide-based polymer.

If so desired, one or more further streams comprising further monomers may be added to the polymerisation feed. Such further monomers are to be polymerisable with lactide. Examples of further monomers include further lactide feeds, but also substituted dilactones, glycolide and caprolactone monomers. In one embodiment, a further lactide stream is provided to the polymerisation feed. By selecting the stereochemical composition of the further lactide feed, the stereochemistry of the final polymer can be tailored.

In one embodiment, the invention pertains to a process for manufacturing a lactide-based polymer comprising the steps of:
providing a lactide stream comprising 50-85 wt. % of meso-lactide and 50-15 wt. % of L-lactide to a polymerisation feed, in particular a feed resulting from the joint crystallisation step described herein,
providing a lactide stream comprising at least 75 wt. % of L-lactide, calculated on total lactide species in the stream, to form a lactide stream comprising 70-99 wt. % L-lactide and 1-30 wt. % meso-lactide, both calculated on total lactide species to a polymerisation feed, the ratios of the streams being such that the polymerisation feed comprises 70-99 wt. % L-lactide and 1-30 wt. % meso-lactide,
and subjecting the polymerisation feed comprising 70-99 wt. % L-lactide and 1-30 wt. % meso-lactide to a polymerisation step to form polylactic acid.

In one embodiment, the invention pertains to a process for manufacturing polylactic acid comprising the steps of
reacting lactic acid to form lactide,
generating a lactide feed comprising lactide and non-lactide contaminants, the lactide comprising 50-85 wt. % of meso-lactide and 50-15 wt. % of L-lactide, and subjecting said feed to a joint crystallisation step to generate a stream containing non-lactide contaminants and a lactide stream reduced in non-lactide contaminants, which lactide stream comprises 50-85 wt. % of meso-lactide and 50-15 wt. % of L-lactide,
combining a lactide stream comprising 50-85 wt. % of meso-lactide and 50-15 wt. % of L-lactide derived from the joint crystallisation step with a lactide stream comprising at least 75 wt. % of L-lactide, calculated on total lactide species in the stream, to form a lactide stream comprising 70-99 wt. % L-lactide and 1-30 wt. % meso-lactide, both calculated on total lactide species,
and subjecting the lactide stream comprising 70-99 wt. % L-lactide and 1-30 wt. % meso-lactide to a polymerisation step to form polylactic acid.

FIG. 4 illustrates an embodiment of this polymerisation process. FIG. 4 is a variation on FIG. 1. In FIG. 4 the purified mixture of 50-85 wt. % of meso-lactide and 50-15 wt. % of L-lactide which is crystallised in crystallisation unit (6) and withdrawn through line (7) is provided to a polymerisation unit (30) where it is combined with L-lactide formed in crystallisation unit (13) and removed through line (14). By selecting the ratios between the two streams, the amount of meso-lactide in the polymerisation reaction may be tailored. The PLA is removed through line (31).

The polymerisation of lactide to form polylactic acid is well known in the art, and requires no further elucidation here. Polylactide generally has a number average molecular weight of above 5 kg/mol, in particular above 10 kg/mol. A value of 100 kg/mol may be mentioned as a maximum.

In a further embodiment, the invention pertains to a process for producing lactic acid wherein at least a portion of the stream comprising meso-lactide and L-lactide and reduced in non-lactide contaminants obtained as described above is hydrolysed to form lactic acid. This allows the manufacture of high-purity lactic acid by processing what in conventional operations is often regarded as remnant streams. The lactic acid thus produced, which has a high purity can be used in many applications. It may, e.g., be used in food applications, or provided, directly or indirectly, to a condensation oligomerisation step or a depolymerisation step. It may, e.g., be used as a starting material in the manufacture of lactate esters. The hydrolysation step may be carried out by methods known in the art. It encompasses in general the reaction of lactide with water.

Also disclosed herein is a racemic mixture of (S)-lactic acid and (R)-lactic acid obtainable by the process for producing a racemic mixture of (S)-lactic acid and (R)-lactic acid according to the invention.

The present application is drafted for a method for processing a lactide feed comprising L-lactide as the predominant lactide isomer. It will however be clear to the skilled person that the same process as described in the present application applies to a lactide feed comprising D-lactide as the predominant lactide isomer, and that where the term “L-lactide” is used in the present application this could be replaced by “D-lactide” and vice versa. It will be clear to the skilled person that such a process of processing a lactide feed comprising D-lactide as the predominant isomer also falls within the scope of the present application.

As will be evident to the skilled person, different embodiments of the present invention can be combined unless they are mutually exclusive.

All percentages used herein are weight percentages, unless specified otherwise.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

The present invention will be elucidate by the following examples, without being limited thereto or thereby.

### Example 1: Proof of Principle, Joint Crystallisation in the Presence of Acetone as a Solvent

A feed mixture with a meso-lactide content of 60.7 wt % and a free acid content of 64.2 meq/kg was subjected to eutectic solution crystallisation in the presence of acetone as solvent. The total starting mass was 200.0 grams. In addition to lactides the starting mixture contained other components. The other components include lactic acid, oligomers of lactic acid and other organic acids. The feed composition is provided in the following table.

| | |
|---|---|
| feed meso-lactide content (wt %) | 60.7 |
| feed total-lactide content (wt %) | 91.0 |
| (total of meso, L- and D-lactide) | |
| feed acetone content (wt %) | 5.0 |
| feed other compounds (wt %) | 4.0 |
| Of which oligomers (sum HL2-HL18, wt %) | 1.6 |

The D-lactide content in the feed has not been determined separately as the D-lactide content is minor (in the range of 0.01 to 5 wt. % (based on the total weight of lactide in the feed)) and D-lactide follows L-lactide in the crystallisation step as was already described above. This also applies to the other examples.

In a double-jacketed, temperature controlled vessel the feed composition was heated to a temperature which was such that all lactide dissolved. The mixture was cooled from 55° C. to 27° C. with a cooling rate of 20° C./h after which the cooling rate was decreased to 10° C./h. Meso-lactide seeding crystals were added after each additional degree of cooling. At 21° C. the seeding crystals did no longer dissolve, and the mixture was kept at that temperature for 30 minutes, in order to provide sufficient time for the crystals to grow. The crystals were isolated through centrifugation. The crystals had a meso-lactide content of 68 wt. %, and a total of L- and D-lactide of 31 wt. %. The meso-lactide content of 68 wt. % is close to the eutectic concentration of meso-lactide indicating both meso-lactide and L- and D-lactide are crystallizing at the eutectic composition. The following table gives the free acid content of the feed and the product, and the reduction factor. The amount of oligomer in feed and product and the associated reduction therein is also provided.

| | |
|---|---|
| feed - free acid (meq/kg) | 64.2 |
| product - free acid (meq/kg) | 16.8 |
| reduction factor | 3.8 |
| feed - oligomers (sum HL2-HL18, wt %) | 1.6 |
| product - oligomers (sum HL2-HL18, wt %) | 0.6 |
| reduction factor | 2.7 |

The reduction factor of 3.8 in free acidity shows that the crystallisation step resulted in an effective separation of the lactide and the acidic components. The amount of oligomers is also substantially reduced, with a reduction factor of 2.7. Overall 21% of the total amount of lactides was recovered as purified crystals in a single pass. The use of further passes would lead to an increase in yield.

### Example 2: Joint Crystallisation in the Presence of Acetone as a Solvent, Lower Crystallization Temperature

A feed mixture with a meso-lactide content of 62.8 wt % and a free acid content of 72.6 meq/kg was subjected to eutectic solution crystallisation in the presence of acetone as solvent. The total starting mass was 204.0 grams. The other components include lactic acid, oligomers of lactic acid and other organic acids. The feed composition is provided in the following table.

| | |
|---|---|
| feed meso-lactide content (wt %) | 62.8 |
| feed total-lactides content (wt %) | 92.1 |
| (total of meso, L- and D-lactide) | |
| feed acetone content (wt %) | 5.0 |
| feed other compounds (wt %) | 2.9 |
| Of which oligomers (sum HL2-HL18, wt %) | 2.3 |

In a double-jacketed, temperature controlled vessel the feed composition was heated to a temperature which was such that all lactide dissolved. The mixture was cooled from 55° C. to 27° C. with a cooling rate of 20° C./h after which the cooling rate was decreased to 10° C./h. Meso-lactide seeding crystals were added after each additional degree of cooling. At 19° C. the seeding crystals did no longer dissolve, and the mixture was kept at that temperature for 30 minutes, in order to provide sufficient time for the crystals to grow. The crystals were isolated through centrifugation.

The crystals had a meso-lactide content of 66 wt. %, and a total of L- and D-lactide of 33 wt. %. The meso-lactide content of 66 wt. % is close to the eutectic concentration of meso-lactide indicating both meso-lactide and L-lactide are crystallizing at the eutectic composition. The following table gives the free acid content of the feed and the product, and the reduction factor. The following table gives the free acid content of the feed and the product, and the reduction factor. The amount of oligomer in feed and product and the associated reduction therein is also provided.

| | |
|---|---|
| feed - free acid (meq/kg) | 72.6 |
| product - free acid (meq/kg) | 25.6 |
| reduction factor | 2.8 |
| feed - oligomers (sum HL2-HL18, wt %) | 2.3 |
| product - oligomers (sum HL2-HL18, wt %) | 0.7 |
| reduction factor | 4 |

The reduction factor of 2.8 shows that the crystallisation step resulted in an effective separation of the lactide and the acidic components. The amount of oligomers is also substantially reduced, with a reduction factor of 4.

Due to the lower crystallization temperature, the amount of crystals formed was higher than in Example 2. In this experiment 34% of the total amount of lactides was recovered as purified crystals. This experiment shows that the recovery of lactides can be controlled.

### Examples 3 and 4: Joint Crystallisation without the Use of a Solvent

Two experiments were conducted without the use of a solvent. The composition of the starting mixtures are given below. The free acidity of the experiment 3 and 4 were 238.8 and 319.9 meq/kg respectively.

| experiment | 3 | 4 |
|---|---|---|
| feed meso-lactide content (wt %) | 58.5 | 62.0 |
| feed total-lactides content (wt %) | 91.4 | 88.3 |
| (total of meso, L- and D-lactide) | | |
| feed other compounds (wt %) | 8.6 | 11.7 |
| Of which oligomers (sum HL2-HL18, wt %) | 1.5 | 2.0 |

In a double-jacketed, temperature controlled vessel the mixtures were heated to a temperature which was such that all lactide was molten. The mixtures were then cooled from ~55° C. to 33° C. with a cooling rate of 20° C./h. After reaching 33° C., the mixture of experiment 3 was cooled to and end temperature of 28° C. with a cooling rate of 10° C./h. After reaching 33° C., the mixture of experiment 4 was cooled to and end temperature of 32° C. with a cooling rate of 10° C./h. In both experiments, upon reaching 33° C. meso-lactide seeding crystals were added after each additional degree of cooling until the seeding crystals no longer dissolved.

Both experiments were stopped when slurries were obtained with about 50% crystal content. The crystals were isolated through centrifugation. The results of the crystallisation are given in the following table.

| experiment | 3 | 4 |
|---|---|---|
| feed - free acid (meq/kg) | 238.8 | 319.9 |
| product - free acid (meq/kg) | 56.8 | 46.6 |
| reduction factor - FA | 4.2 | 6.9 |
| yield (wt. %, calculated on the lactide in the feed) | 43.8 | 31.8 |
| product - meso lactide content (wt. %) | 55.0 | 73.3 |
| product - D and L lactide content (wt. %) | 40.6 | 20.3 |
| product - oligomers | 0.3 | 0.4 |
| reduction factor - oligomers | 4.7 | 4.6 |

These examples show that also for melt crystallisation joint crystallisation can lead to efficient purification, both as regards the reduction of free acids and as regards the reduction factor of 2.8 shows that the crystallisation step resulted in an effective separation of the lactide and the acidic components. The amount of oligomers is also substantially reduced, with a reduction factor of 4.

1. A process for manufacturing lactide with a reduced content of non-lactide contaminants, wherein the process comprises:
providing a lactide feed comprising lactide and non-lactide contaminants, wherein the lactide comprises 50-85 wt. % (calculated on total lactide) of meso-lactide and 50-15 wt. % (calculated on total lactide) of L-lactide; and
subjecting said lactide feed to a joint crystallisation step to generate a liquid stream comprising non-lactide contaminants and a solid lactide stream reduced in non-lactide contaminants, wherein the solid lactide stream comprises 50-85 wt. % (calculated on total lactide) of meso-lactide and 50-15 wt. % (calculated on total lactide) of L-lactide.

2. The process according to claim 1, wherein the feed subjected to the crystallisation step contains 70-99.5 wt. % of total lactide, calculated on the total weight of the feed.

3. The process according to claim 1, wherein the feed subjected to the crystallisation step has a free acid content of at least 30 meq/kg.

4. The process according to claim 1, wherein the feed subjected to the crystallisation step has a meso-lactide content in the range of 55-80 wt. % with the L-lactide content being in the range of 45-20 wt. %.

5. The process according to claim 1, wherein the feed subjected to the joint crystallisation step is at a temperature of at least 50° C.

6. The process according to claim 1, wherein the joint crystallisation is carried out through solution crystallisation.

7. The process according to claim 1, wherein the joint crystallisation is carried out through melt crystallisation.

8. The process according to claim 1, wherein the joint crystallisation step is carried out in 2-10 sequential crystallisation stages.

9. The process according to claim 1, further comprising:
providing a lactide composition comprising L-lactide in an amount of 75-99 wt. % (calculated on total lactide), meso-lactide in an amount of 1-25 wt. % (calculated on total lactide), and non-lactide contaminants, and
generating from the lactide composition the lactide feed comprising lactide and non-lactide contaminants.

10. The process according to claim 9, wherein the lactide composition is prepared by a step comprising reacting lactic acid.

11. The process according to claim 10, wherein the step comprising reacting lactic acid is carried out by a process comprising:
subjecting lactic acid to condensation oligomerisation to form low molecular weight poly(lactic acid), and
depolymerising the low molecular weight poly(lactic acid) to form lactide.

12. The process according to claim 9, wherein the lactide composition is prepared by a process comprising catalytic depolymerisation of polylactide with a number average molecular weight of at least 5 kg/mol to provide the lactide composition.

13. The process according to claim 9, wherein the process further comprises at least one further crystallisation step, wherein the further crystallisation step is selected from
a crystallisation step in which L-lactide is crystallised from a feed and meso-lactide is not,
a crystallisation step in which meso-lactide is crystallised from a feed and L-lactide is not, and
a further joint crystallisation step.

14. The process according to claim 9, further comprising:
subjecting the lactide composition to a separation step to form a first stream having an L-lactide content of at least 80 wt. %, calculated on the total weight of the stream, and a second stream comprising lactide and non-lactide contaminants, wherein the lactide in the second stream comprises 50-85 wt. % of meso-lactide and 50-15 wt. % of L-lactide; and
subjecting the second stream to a joint crystallisation step to generate a stream containing non-lactide contaminants and a solid lactide stream reduced in non-lactide contaminants, wherein the solid lactide stream comprises 50-85 wt. % (calculated on total lactide) of meso-lactide and 50-15 wt. % (calculated on total lactide) of L-lactide.

15. The process according to claim 14, wherein the first stream is subjected to a purification step, in particular a crystallisation step.

16. A process for manufacturing a lactide-based polymer comprising:
providing a lactide stream comprising 50-85 wt. % of meso-lactide and 50-15 wt. % of L-lactide prepared according to claim 1 to a polymerisation feed, and
subjecting the polymerisation feed to a polymerisation step to form a lactide-based polymer.

17. The process according to claim 16, wherein one or more further streams comprising further monomers polymerisable with lactide are added to the polymerisation feed.

18. The process according to claim 17, wherein the one or more further streams is selected from a further lactide stream, a glycolide stream, and a caprolactone stream.

19. The process according to claim 17, comprising:
providing the lactide stream to the polymerisation feed;
providing a lactide stream comprising at least 75 wt. % of L-lactide, calculated on total lactide species in the stream, to form a lactide stream comprising 70-99 wt. % L-lactide and 1-30 wt. % meso-lactide, both calculated on total lactide species to a polymerisation feed, the ratios of the streams being such that the polymerisation feed comprises 70-99 wt. % L-lactide and 1-30 wt. % meso-lactide; and
subjecting the polymerisation feed comprising 70-99 wt. % L-lactide and 1-30 wt. % meso-lactide to a polymerisation step to form polylactic acid.

20. The process according to claim 18, comprising:
reacting lactic acid to form lactide;
generating a lactide feed comprising lactide and non-lactide contaminants, the lactide comprising 50-85 wt. % of meso-lactide and 50-15 wt. % of L-lactide, and subjecting said lactide feed to a joint crystallisation step to generate a stream containing non-lactide contaminants and a lactide stream reduced in non-lactide contaminants, wherein the lactide stream comprises 50-85 wt. % of meso-lactide and 50-15 wt. % of L-lactide;
combining the lactide stream comprising 50-85 wt. % of meso-lactide and 50-15 wt. % of L-lactide derived from the joint crystallisation step with a lactide stream comprising at least 75 wt. % of L-lactide, calculated on total lactide species in the stream, to form a lactide stream comprising 70-99 wt. % L-lactide and 1-30 wt. % meso-lactide, both calculated on total lactide species; and
subjecting the lactide stream comprising 70-99 wt. % L-lactide and 1-30 wt. % meso-lactide to a polymerisation step to form polylactic acid.

21. The process according to claim 1, wherein the solid stream comprising 50-85 wt. % of meso-lactide and 50-15 wt. % of L-lactide is hydrolysed to form lactic acid.
